(19) United States
(12) Patent Application Publication
(10) Pub. No.: US 2015/0313592 A1
(43) Pub. Date: Nov. 5, 2015

(54) Compression Clip Having Convergent Legs

(71) Applicant: IN2BONES, Ecully (FR)

(72) Inventors: Jean-Yves Paul Albert Coillard-Lavirotte, Saint Cyr Au Mont D'or (FR); Stéphane Paul Mauger, Dommiers (FR)

(73) Assignee: IN2BONES, Ecully (FR)

(21) Appl. No.: 14/649,864

(22) PCT Filed: Dec. 6, 2013
(86) PCT No.: PCT/FR2013/052970

§ 371 Date: Jun. 4, 2015

(30) Foreign Application Priority Data
Dec. 6, 2012 (FR) 1261733

(51) Int. Cl. A61B 17/08; A61B 17/10; A61B 17/84

(52) U.S. Cl. CPC A61B 17/083; A61B 17/846; A61B 17/10

(57) ABSTRACT

The invention relates to a medical compression clip (1) capable of imparting compression to two bone fractions and/or to biological tissues, the clip comprising at least two legs (2, 3) extending from a transverse bridge, at least one leg (2) occupying a naturally converging position directed towards the other leg (3), the converging leg (2) being capable of being subjected to temporary elastic deformation and of returning naturally to its converging position, the clip being provided with deformation means enabling a force opposing convergence to be exerted on said at least one converging leg (2, 3) by means of an external tool, the deformation means being formed by a reception member for receiving the external tool, the clip (1) being characterized in that the reception member is arranged within the transverse bridge in such a manner that the external tool can bear against and along said converging leg (2, 3).

Medical clips.

### TECHNICAL FIELD

The present invention relates to the general technical field of medical clips suitable for exerting compression in order to compress together two bone fractions or biological tissue fractions in order to promote osteosynthesis between them, i.e. in order to ensure that they fuse together, in particular with bony fusion, said medical clip having at least two legs that extend longitudinally from a transverse bridge.

The present invention relates to a medical compression clip capable of compressing together two bone fractions and/or tendon or ligament type biological tissue fractions in order to cause them to fuse together, the clip comprising at least two legs extending longitudinally from a transverse bridge, at least one leg occupying a naturally converging position so as to form a converging leg that is directed towards the other leg, the clip being made out of a material presenting sufficient elasticity to enable the converging leg to be subjected to temporary elastic deformation away from its naturally converging position, e.g. in order to enable it to be put into place, and to return naturally towards its naturally converging position in order to exert a compression force, the clip being provided with deformation means enabling a force opposing the convergence of said converging leg to be exerted on said at least one converging leg by means of an external tool co-operating with said deformation means in order to impart temporary deformation to the converging leg, the deformation means being formed by a reception member for receiving an external tool, the reception member being arranged at the junction between said at least one converging leg and the transverse bridge.

The present invention also relates to a kit formed by a medical compression clip and by a tool suitable for co-operating with the clip in order to put it into place.

### PRIOR ART

It is already known to have recourse to medical compression clips for holding bone fragments in contact with one another in order to achieve progressive osteosynthesis between them, i.e. bony fusion by cell growth at the interfaces between the bony fragments.

In conventional manner, the bone fractions that result from accidents that have led to bones breaking are firstly put back into place by a surgeon, and they are then put into contact with one another and held in contact with one another along fracture interfaces by means of clips.

In known manner, clips are generally in the form of two legs extending longitudinally from a transverse bridge so as to form a U-shaped clip. After making two substantially coplanar and parallel holes in respective ones of the bone fractions between which bony fusion is to be achieved, the surgeon inserts each of the legs of the clip in the holes until the transverse bridge comes to rest on the outside surfaces of the bone fractions. This makes it possible to hold the two bone fractions to be fused together in contact for a length of time that is sufficient to allow cell growth and bony fusion to take place.

Clips of the above-mentioned type generally give satisfaction and make it possible to ensure that bony fusion ultimately takes place between the bone fragments.

Nevertheless, it is found that prior clips suffer from a drawback associated with the time needed to achieve complete bony fusion. The time is generally considered as being long and it is associated with the difficulty of putting the two bone fragments into contact with each other with sufficient pressure to encourage rapid growth of bone cells. It is difficult to put conventional prior art clips of U-shape with parallel legs correctly into place and it is also difficult to obtain sufficient contact pressure between the two bone fragments in order to lead rapidly to good bone fusion.

### SUMMARY OF THE INVENTION

Consequently, the objects given to the invention seek to remedy the various above-mentioned known drawbacks of the prior art and to propose a novel medical compression clip that is capable of easily generating compression between bone fractions or biological tissues to be fused one against the other when the clip is inserted in said bone fractions or biological tissues.

Another object of the invention seeks to propose a novel medical compression clip that can easily be put into place by a surgeon, while also being capable of exerting compression on bone fractions.

Another object of the invention seeks to propose a novel medical compression clip that can exert compression on bone fractions to be fused together, while also being particularly robust and presenting good fatigue strength.

Another object of the invention seeks to propose a novel medical compression clip that can be put into place by a surgeon, while not harming the original mechanical properties of the clip.

Another object of the invention seeks to propose a medical device constituted by a clip comprising a clip and a member for putting the clip into place, which member enables a medical compression clip to be put into place easily.

The objects given to the invention are achieved with the help of a medical compression clip capable of compressing together two bone fractions and/or tendon or ligament type biological tissue fractions in order to cause them to fuse together, the clip comprising at least two legs extending longitudinally from a transverse bridge, at least one leg occupying a naturally converging position so as to form a converging leg that is directed towards the other leg, the clip being made out of a material presenting sufficient elasticity to enable the converging leg to be subjected to temporary elastic deformation away from its naturally converging position, e.g. in order to enable it to be put into place, and to return naturally towards its naturally converging position in order to exert a compression force, the clip being provided with deformation means enabling a force opposing the convergence of said converging leg to be exerted on said at least one converging leg by means of an external tool co-operating with said deformation means in order to impart temporary deformation to the converging leg, the deformation means being formed by a reception member for receiving an external tool, the reception member being arranged at the junction between said at least one converging leg and the transverse bridge, the clip being characterized in that the reception member is arranged within the transverse bridge in such a manner that the external tool, of the pin or rod type, can bear against and along said converging leg in order to impart the temporary elastic deformation.

The objects given to the invention are also achieved by a kit constituted by a medical compression clip in accordance with the invention and by a tool suitable for co-operating with the clip to impart temporary elastic deformation thereto.

### BRIEF DESCRIPTION OF THE DRAWINGS

Other features and advantages of the invention appear and can be seen in greater detail on reading the following description made with reference to the accompanying drawings that are given solely by way of non-limiting illustrative example, and in which:

FIG. 1 is a general perspective view from above of a first variant embodiment of a medical compression clip in accordance with the invention;

FIG. 2 is a lateral section view showing a compression clip in accordance with the variant shown in FIG. 1;

FIG. 3 is a perspective view from beneath of a medical compression clip in accordance with the first variant shown in FIG. 1;

FIG. 4 is a perspective view from above of a medical compression clip in accordance with the variant shown in FIG. 1;

FIG. 5 is a section view in perspective of a variant of the compression clip in accordance with the invention;

FIG. 6 is a lateral section view showing a variant of the compression clip in accordance with the invention as shown in FIG. 5;

FIG. 7 is a perspective view showing another variant embodiment of a compression clip in accordance with the invention;

FIG. 8 is a view from above showing a variant embodiment of a compression clip in accordance with the invention as shown in FIG. 7;

FIG. 9 is a diagrammatic view of a kit comprising firstly a medical compression clip in accordance with the invention corresponding to the first variant embodiment as shown in FIGS. 1 to 4, and secondly a tool for putting it into place;

FIG. 10 shows a kit in accordance with the invention formed firstly by a medical compression clip shown in section and in accordance with the second variant embodiment as shown in FIGS. 5 and 6, and secondly a tool suitable for co-operating with the clip; and

FIG. 11 shows a kit formed firstly by a medical compression clip in accordance with the invention and corresponding to another variant embodiment as shown in FIGS. 7 and 8, and secondly by a tool suitable for co-operating with the clip in order to deform it.

### BEST MANNER OF PERFORMING THE INVENTION

As shown in the figures, the invention relates to a medical compression clip 1 capable of compressing two bone fractions and/or two biological tissue fractions such as tendons or ligaments in order to ensure osteosynthesis between them.

Thus, the medical compression clip 1 in accordance with the invention may be put into place in order generally to ensure bone fusion between two bone fractions or fusion between two ligaments or indeed two tendons, or even between a bone fraction and a ligament or a bone fraction and a tendon, without thereby going beyond the ambit of the invention.

As shown generally, the medical compression clip 1 of the invention comprises two legs 2 and 3, each having a distal end 2D, 3D and a proximal end 2P, 3P, which distal ends are connected together by a transverse bridge 4.

Thus, the medical compression clip 1 of the invention has at least two legs 2 and 3 extending longitudinally from the transverse bridge 4 so as to form generally a two-branch U-shaped clip.

According to an important characteristic of the invention, the medical compression clip 1 of the invention has at least one leg 2 that occupies a naturally converging position, thus forming a converging leg 2 that is directed towards the other leg 3, the clip being made of a material that presents sufficient elasticity for the converging leg 2 to be capable of being subjected to temporary elastic deformation away from its naturally converging position, e.g. in order to enable it to be put into place, and to be capable of returning naturally towards its naturally converging position in order to exert a compression force by virtue of its intrinsic elasticity.

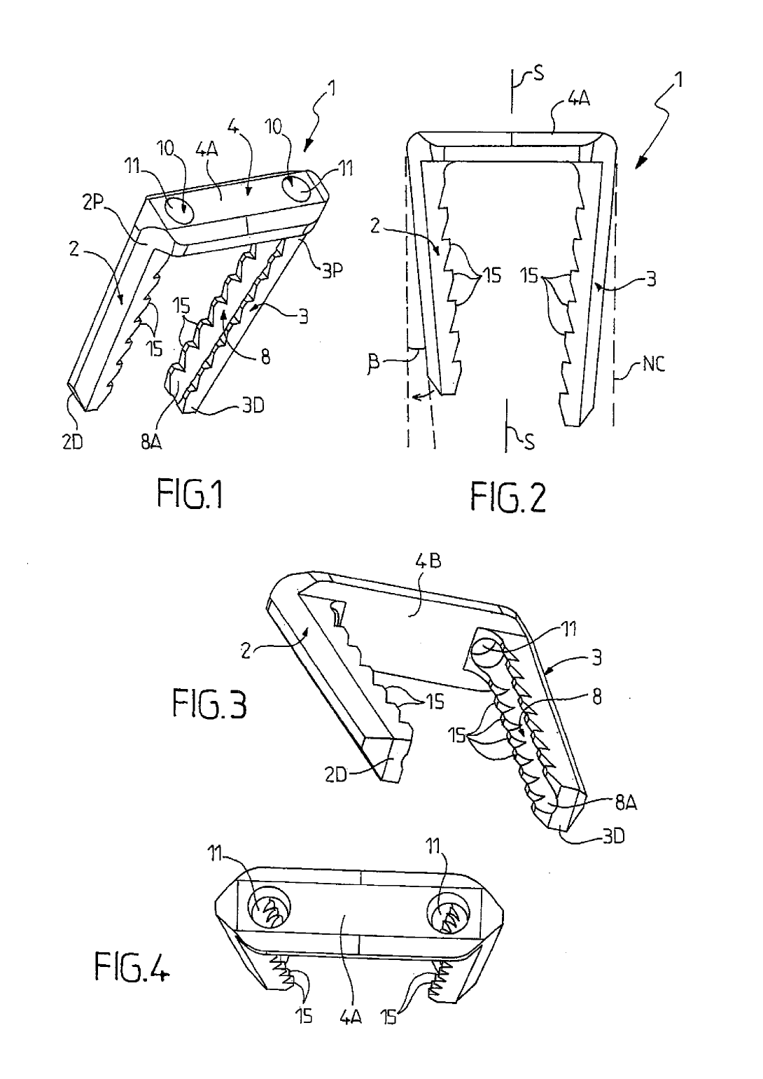

In the variant embodiment shown in FIGS. 1 to 4, the medical compression clip 1 in accordance with the invention is provided with two converging legs 2 and 3 that converge towards each other or that are directed towards a main plane of symmetry S (FIG. 1) of the clip. Nevertheless, the invention relates more generally to a medical compression clip 1 that may have only one converging leg 2, with the other leg 3 naturally (i.e. at rest) occupying a non-converging position such that it occupies a position NC (shown in dashed lines in FIG. 2) in which the leg 3 extends substantially orthogonally to the general plane in which the transverse bridge 4 extends (FIG. 2).

In this position, and in a variant that is not shown in the figures, the non-converging leg 3 then forms, at rest, a right angle with the transverse bridge 4, with only the leg 2 forming a leg that converges at rest.

Thus, without going beyond the ambit of the invention, the medical compression clip 1 in accordance with the invention may include at least one converging leg 2 or two converging legs 2 and 3.

In the description below and in the accompanying drawings, the variant embodiments of the medical compression clip 1 in accordance with the invention are all shown, for simplification purposes, as having two converging legs 2 and 3, without the subject matter of the invention being limited thereby to the presence of two converging legs 2 and 3.

The medical compression clip 1 in accordance with the invention is made from a material such as polyetheretherketone (PEEK) or out of a metal material such as titanium, presenting sufficient elasticity for the converging leg(s) 2, 3 to be deformed or spaced apart from the natural rest position on being subjected to temporary elastic deformation, e.g. in order to reach the position shown in dashed lines in FIG. 2 (corresponding substantially to positions in which the legs 2, 3 are temporarily coplanar and parallel), in order to return subsequently to the initial or original converging position when the spacing-apart force ceases, in order to exert compression. In addition, in order to enable the surgeon to deform the clip easily, the latter has deformation means 10 suitable for exerting a force opposing the convergence of said converging leg 2 in order to deform it or move it away temporarily, this force being exerted on said at least one converging leg 2, 3 by means of an external tool 20 that co-operates with said deformation means 10.

Thus, a medical compression clip 1 is made available that enables the surgeon to deform clips in accordance with the invention very easily and in temporary manner so as to make the legs 2 and 3 substantially coplanar and parallel, and then put the legs 2 and 3 into holes previously formed in the fragments of bone or biological tissue to be fused together, after which the spacing-apart force can be released so as to allow the clip to close, thereby compressing the two fragments of bone and/or biological tissue such as tendons or ligaments.

As shown in the figures, the deformation means 10 are formed by a reception member for receiving the external tool 20, the reception member being arranged within the transverse bridge 4 at the junction between said at least one converging leg 2, 3 and the transverse bridge 4.

Advantageously, the reception member is formed by at least one through orifice 11 formed through the thickness of the transverse bridge 4, as shown in the variant of FIGS. 1 to 4, or by at least one open cutout 12 (FIGS. 7, 8, 11) formed in the thickness of the transverse bridge 4 so as to open out substantially in register with or plumb with said converging leg 2, 3 so that the external tool 20 of the pin or rod type can be passed through the orifice 11 or the open cutout and press against and along said converging leg 2, 3 in order to impart the temporary elastic deformation when the external tool 20 is used.

Thus, each through orifice 11 is formed through the thickness of the transverse bridge 4 so as to open out in the vicinity of the proximal end 2P and/or 3P of each converging leg 2, 3 and at the peripheries of said legs.

In this way, the external tool 20, such as a pin 21, can be engaged axially in said through orifice 11 and can slide, e.g. along the inside face 8 of the leg 2, 3, e.g. at least as far as the distal end 2D, 3D.

This arrangement enables the surgeon to exert an elastic deformation force on each converging leg 2, 3 in question by causing the pin 21, that bears against fractions of the inside faces 8 of the through orifice 11 or of the open cutout 12, to pivot towards the inside of the clip, i.e. towards the plane of symmetry S.

In order to provide a bearing area that facilitates deformation without that creating fatigue zones within the clip, each converging leg 2, 3 is advantageously provided on its inside face 8 with a reception groove 8A for receiving the external tool 20, which groove is of section that is complementary to the section of the external tool, said groove 8A extending the through orifice 11 or the open cutout 12.

As shown, and solely as a variant, the groove 8A presents a cross-section that is curved and of size similar to the cylindrical section of the external member 20.

In particularly advantageous manner and in order to reinforce the robustness of the clip and enable the converging legs 2, 3 to be deformed elastically while generating minimum fatigue in the material constituting the clip, the through orifice 11 or the open cutouts 12 are shaped so as to enable the external tool 20 to bear linearly while imparting the temporary elastic deformation.

In a particularly advantageous variant of the invention as shown in FIGS. 6, 5, and 10, the through orifice 11 then has a conic cross-section 17. In similar manner, the open cutout 12 also has a semi-conic cross-section.

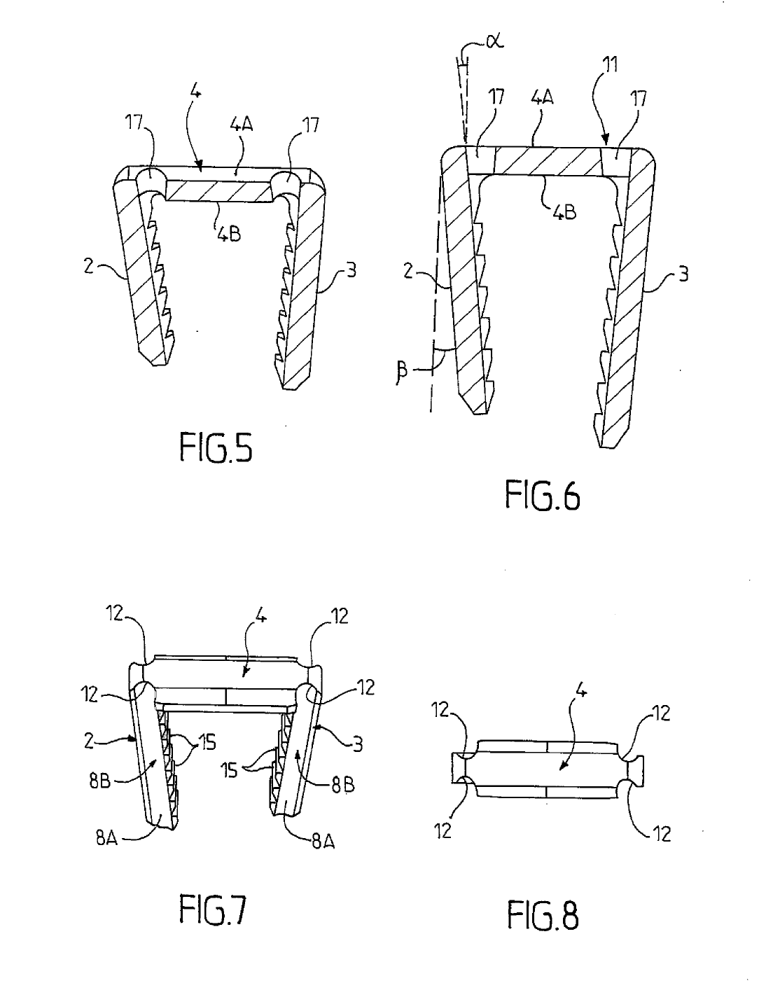

As shown in FIGS. 2 and 6, for example, the conic section 17 of the through orifices 11 or the semi-conic section of the open cutout 12 is arranged so that the larger diameter is flush with the top face 4A of the transverse bridge 4 and the smaller diameter is flush with the bottom face 4B.

The same arrangement is used for the semi-conic cross-section of the open cutout 12 where the larger half-diameter is flush with the top surface 4A.

Because of this geometrical arrangement, the external member 20 comes to bear in linear manner solely against a fraction of the larger diameter or a fraction of the larger half-diameter as defined above, thereby greatly reducing the forces exerted on the converging leg while it is being spaced outwards, thereby preserving its elasticity.

According to a particularly advantageous characteristic of the invention, the cone angle α of the conic cross-section or of the semi-conic cross-section is substantially equal to the angle of convergence β of said at least one converging leg 2, 3.

The convergent angle β and the cone angle α may for example lie in the range 2 degrees to 20 degrees, e.g.

being about 5 degrees.

By selecting these specific angles, it is possible to avoid creating stress zones in the shoulders of the clip.

According to another advantageous characteristic of the clip of the invention, the transverse bridge 4 is substantially curved when the clip is in its rest configuration so as to enable it to be rectilinear when the legs 2 and 3 are substantially mutually parallel and coplanar.

In this variant embodiment (not shown in the figures), the curvature of the transverse bridge 4 presents, at rest, a bottom face 4B that is concave, with the top face 4A then being convex.

As also shown in the figures, the legs 2 and 3 of the clip are advantageously of different lengths, the leg 2 being shorter than the leg 3, for example. This arrangement makes it easier for the surgeon to insert the legs 2 and 3. Nevertheless, it is possible for the legs 2 and 3 to be of equal length.

Thus, in the preferred variant embodiments shown in FIGS. 1 to 11, the clip of the invention comprises:
at least two converging legs 2, 3 that converge towards each other; and
at least two through orifices 11 or at least two open cutouts 12.

In the variant shown in FIGS. 7 to 11, the compression clip of the invention has two pairs of open cutouts 12, the open cutouts 12 of a pair being arranged facing each other on either sides of the transverse bridge 4 so as to open out perpendicularly to the side faces 8B of said converging legs 2, 3.

FIGS. 7 and 8 indeed show an embodiment variant in which the open cutouts 12 are formed at both opposite ends of the transverse bridge 4 and extend the grooves 8A such that in this embodiment variant the medical compression clip 1 in accordance with the invention has four open cutouts 12 and four grooves 8A that are associated in pairs with each converging leg 2, 3.

It is also possible to envisage making a clip of the invention with three or four or even more converging legs without going beyond the ambit of the invention.

As shown in the figures, each leg may be provided with a set of teeth 15 forming a double serie of teeth 15, e.g. situated on either side of the groove 8A with the teeth being arranged one above another along the entire length of the converging leg(s) 2, 3.

The teeth 15 enable the clip to be held better in place, and in particular they avoid the clip escaping from the housings in which it is inserted within the bone mass or the biological tissue.

The present invention also relates to a kit formed firstly by a medical compression clip 1 as described above and secondly by an external tool 20 suitable for co-operating with the clip in order to deform it elastically and temporarily.

In an advantageous version of the invention, the kit including the medical compression clip 1 as described above constitutes a medical device and it may be formed by at least one external tool 20 of the pin or rod type 21 that is to co-operate with the deformation means 10 for deforming the clip in order to deform it elastically and temporarily.

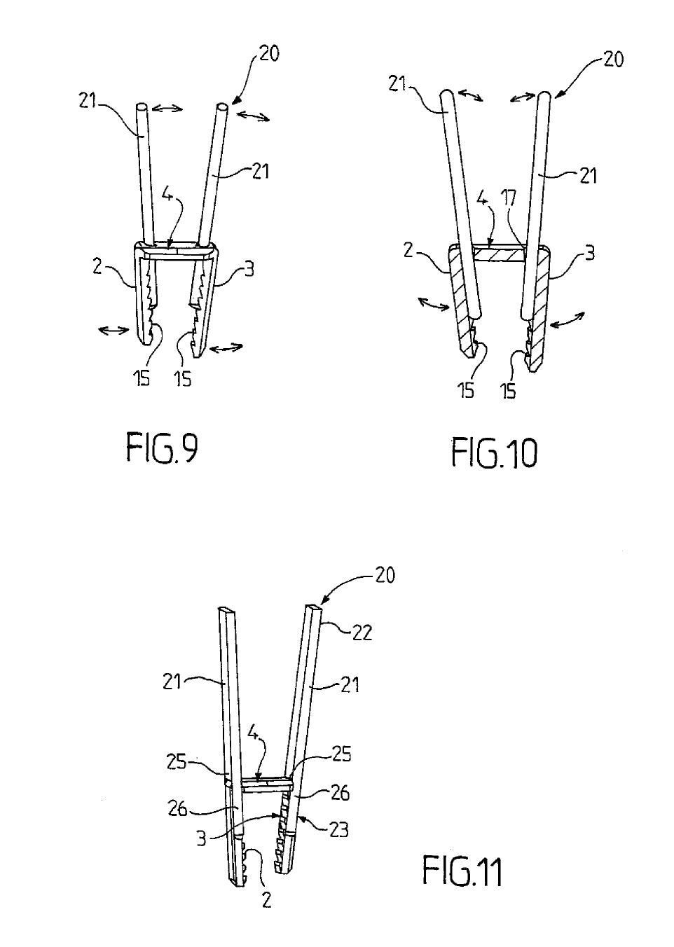

As shown in FIGS. 9, 10, and 11, the external tool 20 comprises at least one pin 21, and preferably two pins 21 for engaging in the through orifices 11 or in the open cutouts 12 so as to impart temporary elastic deformation to the converging leg 2, 3 in question of the clip.

The pins 21 or rods in accordance with the invention may be made of metal materials and they may be of any section, preferably of circular section, and in any event of a section that matches the section of the groove 8A so as to be able to bear over an area on the converging leg(s) 2, 3.

In order to put the clip of the embodiment variant shown in FIGS. 7 and 8 into place, the external tool 20 is advantageously provided with a grip end 22 opposite to a U-shaped end 23 having a core 25 and two branches 26, the core 25 being for placing over the transverse bridge 4 when the branches 26 are inserted in the open cutouts 12 in order to impart temporary elastic deformation to the converging leg(s) 2, 3 of the clip in question. In this situation (FIG. 11), the branches 26 are engaged in the grooves 8A and bear over an area within the grooves 8A formed in the lateral faces 8B of the converging legs 2, 3.

The branches 26 of the external tool 20 are preferably designed to be inserted in the open cutouts 12 so that said branches 26 are embedded in the converging leg(s) 2, 3, the open cutouts 12 being designed to surround said branches 26, at least in part.

The invention also provides a method of implanting a medical compression clip in accordance with the invention, the method comprising the following successive steps:
after initially making coplanar and parallel holes suitable for receiving the legs 2, 3 of the clip 1 in the bone fractions and/or in the tendon or ligament type biological tissues that are to be compressed against each other by the clip 1;
temporarily deforming the converging leg(s) 2, 3 of the clip 1 away from their naturally converging position so that the legs 2, 3 occupy a position in which they are substantially parallel and lie in the same plane;
while keeping the converging leg(s) 2, 3 in this stressed position, inserting the legs 2, 3 in the bone fractions and/or the tendon or ligament type biological tissues that are to be compressed together by the clip 1, and in particular inserting them in the holes; and
then releasing the legs (2, 3) so that the converging leg(s) (2, 3) reclose(s) and provide(s) compression.

In the invention, the temporary deformation of the converging leg(s) 2, 3 is thus imparted by moving the converging legs 2, 3 apart so as to cause them to occupy a position in which they are substantially orthogonal or perpendicular to the extension plane that contains the transverse bridge 4.

In this position, the clip can easily be inserted in the holes previously made by the surgeon, and insertion can be made even easier if the clip has two converging legs 2, 3 of unequal lengths.

In this situation, the surgeon can indeed begin by inserting one leg only in its hole without it being immediately necessary to bring both converging legs 2 and 3 into alignment facing each of the holes.

In the invention, deformation is performed with the help of an external tool 20 that bears against the clip and on the inner or outer surfaces of the converging legs 2, 3 in order to space them apart.

Finally, in the invention, the external tool 20 is engaged in through orifices 11 or in open cutouts 12 formed in the clip so as to bear all along the converging leg(s) 2, 3 in order to impart temporary elastic deformation to the leg in question of the clip.

In the invention, once the surgeon releases the force exerted on the external tools 20, the medical compression clip 1 in accordance with the invention immediately recloses because of the intrinsic internal elasticity of each of the converging legs 2, 3, thereby contributing to put the two bone fragments or the biological tissues immediately into compression.

### SUSCEPTIBILITY OF INDUSTRIAL APPLICATION

The invention finds its industrial application in the design, the implementation, and the fabrication of medical clips for compressing bone fragments and/or biological tissues.

1. A medical compression clip (1) capable of compressing together two bone fractions and/or tendon or ligament type biological tissue fractions in order to cause them to fuse together, the clip comprising at least two legs (2, 3) extending longitudinally from a transverse bridge (4), at least one leg (2) occupying a naturally converging position so as to form a converging leg (2) that is directed towards the other leg (3), the clip being made out of a material presenting sufficient elasticity to enable the converging leg (2) to be subjected to temporary elastic deformation away from its naturally converging position, e.g. in order to enable it to be put into place, and to return naturally towards or to its naturally converging position in order to exert a compression force, the clip being provided with deformation means (10) enabling a force opposing the convergence of said converging leg (2, 3) to be exerted on said at least one converging leg (2, 3) by means of an external tool (20) co-operating with said deformation means (10) in order to impart temporary deformation to the converging the deformation means (10) being formed by a reception member for receiving an external tool (20), the reception member being arranged at the junction between said at least one converging leg (2, 3) and the transverse bridge (4), the clip (1) being characterized in that the reception member is arranged within the transverse bridge (4) in such a manner that the external tool, of the pin or rod type (21), can bear against and along said converging leg (2, 3) in order to impart the temporary elastic deformation.

2. A clip according to claim 1, in which the reception member is formed by a through orifice (11) formed through the thickness of the transverse bridge (4) so as to open out substantially plumb with said converging leg (2, 3) so that the external tool (20), of the pin or rod type (21), can be engaged through the through orifice (11) and bear against and along said converging leg (2, 3) in order to impart the temporary elastic deformation.

3. A clip according to claim 2, in which said at least one converging leg (2, 3) is provided, on its inside face (8) or on its lateral face (8B), with a groove (8A) for receiving the external tool (20), the groove being of section that is complementary to the section of the external tool (20), said groove (8A) extending the through orifice (11).

4. A clip according to claim 2, in which the through orifice (11) is shaped so as to enable the external tool (20) to bear linearly while imparting the temporary elastic deformation.

5. A clip according to claim 4, in which the through orifice (11) has a conic cross-section.

6. A clip according to claim 5, in which the cone angle α of the conic cross-section is equal to the angle of convergence β of said at least one converging leg (2, 3).

7. A clip according to claim 1 in which said clip includes:
at least two converging legs (23) of convergence directed towards each other; and at least two through orifices (11) formed through the transverse bridge.

8. A clip according to claim 1, in which the reception member is formed by an open cutout (12) formed in the thickness of the transverse bridge (4) so that the external tool (20), of pin or rod type (21), can be engaged in the open cutout (12) and come to bear against and along said converging leg (2, 3) in order to impart the temporary elastic deformation.

9. A clip according to claim 8, in which said at least one converging leg (2, 3) is provided, on its inside face (8) or its lateral face (8B), with a groove (8A) for receiving the external tool (20), the groove being of section that is complementary to the section of the external tool (20), said groove (8A) extending the open cutout (12).

10. A clip according to claim 8 in which the open cutout (12) is shaped to enable the external tool (20) to bear in linear manner when imparting the temporary elastic deformation.

11. A clip according to claim 10, in which the open cutout (12) is of semi-conic cross-section.

12. A clip according to claim 11, in which the cone angle α of the semi-conic cross-section is equal to the angle of convergence β of said at least one converging leg (2, 3).

13. A clip according to claim 1 in which said clip also comprises:
at least two converging legs (2, 3) that converge towards each other; and
at least two open cutouts (12) in said transverse bridge.

14. A clip according to claim 13, in which said clip includes two pairs of open cutouts (12), the cutouts of a given pair being arranged facing each other on either side of the transverse bridge (4).

15. A clip according to claim 1 in which the transverse bridge (4) is curved when the clip is in its rest configuration so as to enable it to be rectilinear when the legs (2, 3) are substantially mutually parallel and coplanar.

16. A clip according to claim 1 in which the legs of the clip are of unequal lengths.

17. A clip according to claim 1 in which said clip is made of polyetheretherketone (PEEK).

18. A kit formed by a medical compression clip (1) according to claim 1 and by at least one external tool (20) for co-operating with the deformation means (10) of the clip in order to impart temporary elastic deformation thereto.

19. A kit according to claim 1 in which the external tool (20) comprises at least one pin (21), and preferably two pins, for engaging through the through orifices (11) or in the open cutouts (12) in order to impart the temporary elastic deformation to the converging leg (2, 3) in question of the clip.

20. A kit according to claim 19, in which the external tool (20) is provided with a U-shaped end having a core (25) and two branches (26), the core (25) being designed to be placed over the transverse bridge (4) and the branches (26) to be inserted in the open cutouts (12) in order to impart the temporary elastic deformation to the converging leg(s) (2, 3) in question of the clip.

21. A kit according to claim 20, in which the branches (26) of the external tool (20) are designed to be inserted in the open cutouts (12) in such a manner that said branches (26) are embedded in the converging leg(s) (2, 3).

22. A method of implanting a medical compression clip as defined in claim 1, the method comprising the following successive steps:
temporarily deforming the converging leg(s) (2, 3) of the clip (1) away from their naturally converging position;
while keeping the converging leg(s) (2, 3) in this stress position, inserting said legs (2, 3) in the bone fractions and/or in the tendon or ligament type biological tissues that are to be put into compression by the clip (1); and
releasing the legs (2, 3) so that the converging leg(s) (2, 3) reclose(s) and provide(s) compression.

23. A method according to claim 22, in which the legs (2, 3) are inserted in the bone fractions and/or in the tendon or ligament type biological tissues that are to be put into compression by the clip (1) after previously forming coplanar and parallel holes suitable for receiving the legs (2, 3) of the clip (1) in the bone fractions and/or in the tendon or ligament type biological tissues that are to be put into compression by the clip (1).

24. A method according to claim 22 in which the converging leg(s) (2, 3) of the clip (1) is/are subjected to temporary deformation away from their naturally converging position until the leg(s) (2 3) occupy a position in which they are substantially parallel and lie in the same plane.
